(19) United States
(12) Patent Application Publication
(10) Pub. No.: US 2024/0152778 A1
(43) Pub. Date: May 9, 2024

(54) MODEL GENERATION APPARATUS, MODEL GENERATION METHOD, CONDITION PREDICTION APPARATUS, CONDITION PREDICTION METHOD, AND RECORDING MEDIUM

(71) Applicant: NEC Corporation, Minato-ku, Tokyo (JP)

(72) Inventors: Yuki Kosaka, Tokyo (JP); Kenji Araki, Tokyo (JP)

(73) Assignee: NEC Corporation, Minato-ku, Tokyo (JP)

(21) Appl. No.: 18/272,286

(22) PCT Filed: Jan. 14, 2021
(86) PCT No.: PCT/JP2021/001011

§ 371 Date: Jul. 13, 2023

(51) Int. Cl. G06N 5/022

(52) U.S. Cl. CPC G06N 5/022

(57) ABSTRACT

A model generation apparatus includes: an acquisition unit for obtaining learning data including a first index value indicating a condition of a sample patient at a first time, and a second index value indicating a condition of the sample patient at a second time that is after the first time; and a learning unit for learning a prediction mode for predicting a condition of a target patient at the second time on the basis of a condition of the target patient at the first time, wherein the learning unit is configured to learn the prediction model by updating the prediction model on the basis of a third index value indicating a condition of the sample patient at the second time that is predicted by the prediction model on the basis of the first index value, and a change information indicating a change tendency of the condition over time.

### TECHNICAL FIELD

The present invention relates to technical fields of a model generation apparatus, a model generation method and a recording medium that are configured to generate a prediction model for predicting a condition of a patient, and a condition prediction apparatus, a condition prediction method, and a recording medium that are configured to predict a condition of a patient by using the prediction model.

### BACKGROUND ART

An apparatus that predicts the condition of a patient in a facility such as a hospital is known. For example, Patent literature 1 describes an apparatus that predicts a degree of recovery of the condition of a disease of a patient, in order to match the patient and a facility that accepts the patient.

In addition, as a background art document related to the present application, Non-Patent literature 1 is cited.

### CITATION LIST

### Patent Literature

Patent Literature 1: International Publication No. 2020/071540 pamphlet

### Non-Patent Literature

Non-Patent Literature 1: Tetsuo Oyama, “Outcome Prediction for Stroke Patients: An Application for Local Health care Networking”, The 3rd Annual Meeting of the Japanese Association of Rehabilitation Medicine, Jpn. J. Rehabil. Med. 2009, pp108-117, 2008

### SUMMARY OF INVENTION

### Technical Problem Solved by Invention

In order to predict the condition of a patient at one time, a prediction model that predicts the condition of the patient at the one time on the basis of the condition of the patient at another time that is different from the one time may be used. When such a prediction model is used, in order to improve the prediction accuracy of the prediction model, it is preferable to perform a learning operation of updating the prediction model (e.g., updating a parameter of the prediction model) by using learning data including the actual condition of the patient at one time and the actual condition of the patient at another time. On the other hand, in order to sufficiently improve the prediction accuracy of the prediction model, a certain amount of learning data are required. In order to generate the learning data, however, it is necessary to actually measure the condition of the patient. Therefore, it is not always possible to prepare a sufficient amount of learning data. As a result, it is hardly possible to properly improve the prediction accuracy of the prediction model, and consequently, it is hardly possible to properly predict the condition of the patient.

It is therefore an example object of the present invention to provide a model generation apparatus, a model generation method, a condition prediction apparatus, a condition prediction method, and a recording medium that are capable of solving the above-described technical problems. As an example, it is an example object of the present invention to provide a model generation apparatus, a model generation method, and a recording medium that are configured to properly improve the prediction accuracy of a prediction model, and a condition prediction apparatus, a condition prediction method, and a recording medium that are configured to properly predict the condition of a patient.

### Solution to Problem

A model generation apparatus according to an example aspect includes: an acquisition unit that is configured to obtain learning data including a first index value indicating a condition of a sample patient at a first time, and a second index value indicating a condition of the sample patient at a second time that is after the first time; and a learning unit that is configured to learn a prediction model for predicting a condition of a target patient at the second time on the basis of a condition of the target patient at the first time, wherein the learning unit is configured to learn the prediction model on the basis of a third index value indicating a condition of the sample patient at the second time that is predicted by the prediction model on the basis of the first index value, and a change information indicating a change tendency of the condition over time.

A model generation method according to an example aspect includes: obtaining learning data including a first index value indicating a condition of a sample patient at a first time, and a second index value indicating a condition of the sample patient at a second time that is after the first time; and learning a prediction model for predicting a condition of a target patient at the second time on the basis of a condition of the target patient at the first time, wherein in the learning of the learning, the prediction model is learned on the basis of a third index value indicating a condition of the sample patient at the second time that is predicted by the prediction model on the basis of the first index value, and a change information indicating a change tendency of the condition over time.

A recording medium according to a first example aspect is a recording medium on which a computer program that allows a computer to execute a model generation method is recorded, the model generation method including: obtaining learning data including a first index value indicating a condition of a sample patient at a first time, and a second index value indicating a condition of the sample patient at a second time that is after the first time; and learning a prediction model for predicting a condition of a target patient at the second time on the basis of a condition of the target patient at the first time, wherein in the learning of the learning, the prediction model is learned on the basis of a third index value indicating a condition of the sample patient at the second time that is predicted by the prediction model on the basis of the first index value, and a change information indicating a change tendency of the condition over time.

A condition prediction apparatus according to an example aspect includes: a condition acquisition unit that is configured to obtain a condition information about a condition of a target patient at a first time; and a prediction unit that is configured to predict a condition of the target patient at a second time that is after the first time, by using the condition information and a prediction model for predicting a condition of the target patient at the second time on the basis of the condition of the target patient at the first time, wherein the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

A condition prediction method according to an example aspect includes: obtaining a condition information about a condition of a target patient at a first time; and predicting a condition of the target patient at a second time that is after the first time, by using the condition information and a prediction model for predicting a condition of the target patient at the second time on the basis of the condition of the target patient at the first time, wherein the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

A recording medium according to a second example aspect is a recording medium on which a computer program that allows a computer to execute a condition prediction method is recorded, the condition prediction method including: obtaining a condition information about a condition of a target patient at a first time; and predicting a condition of the target patient at a second time that is after the first time, by using the condition information and a prediction model for predicting a condition of the target patient at the second time on the basis of the condition of the target patient at the first time, wherein the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

### Effect of the Invention

According to the model generation apparatus in the example aspect, the model generation method in the example aspect, and the recording medium in the first example aspect, it is possible to properly improve the prediction accuracy of the prediction model. Furthermore, according to the condition prediction apparatus in the example aspect, the condition prediction method in the example aspect, and the recording medium in the second example aspect, it is possible to properly predict the condition of a patient.

### BRIEF DESCRIPTION OF DRAWINGS

FIG. 1 is a block diagram illustrating a configuration of a model generation apparatus according to an example embodiment.

FIG. 2 is a data structure diagram illustrating a data structure of a learning data set according to the example embodiment.

FIG. 3 is a flowchart illustrating a flow of a model generation operation performed by the model generation apparatus according to the example embodiment.

FIG. 4 is a graph illustrating an example of a change curve indicating a change tendency of an FIM score.

FIG. 5 is a block diagram illustrating a configuration of a condition prediction apparatus according to the example embodiment.

FIG. 6 is a flowchart illustrating a flow of a condition prediction operation performed by the condition prediction apparatus according to the example embodiment.

FIG. 7 is a graph illustrating the prediction accuracy of a prediction model generated by the model generation operation in the example embodiment and the prediction accuracy of a prediction model generated by a model generation operation in a comparative example.

### DESCRIPTION OF EXAMPLE EMBODIMENT

Hereinafter, with reference to the drawings, a model generation apparatus, a model generation method, a condition prediction apparatus, a condition prediction method, and a recording medium according to an example embodiment will be described. The following describes, in order, a model generation apparatus 1 to which the model generation apparatus, the model generation method, and the recording medium according to the example embodiment are applied, and a condition prediction apparatus 2 to which the condition prediction apparatus, the condition prediction method, and the recording medium according to the example embodiment are applied.

### (1) Model Generation Apparatus 1

First, the model generation apparatus 1 will be described. The model generation apparatus 1 is an apparatus that is configured to perform a model generation operation. The model generation operation is an operation for generating a prediction model M. The prediction model M is a model that predicts, on the basis of a condition of a patient at a time tA, each of a condition of the patient at a time tB that is after the time tA and a condition of the patient at a time tC that is after the time tB. The prediction model M may separately include a model that predicts the condition of the patient at the time tB that is after the time tA on the basis of the condition of the patient at the time tA, and a model that predicts the condition of the patient at the time tC that is after the time tB on the basis of the condition of the patient at the time tA. Alternatively, the prediction model M may be a single model that predicts each of the condition of the patient at the time tB and the condition of the patient at the time tC that are after the time tA, on the basis of the condition of the patient at the time tA. The example embodiment describes an example in which the prediction model M is a model that allows the output of an index value indicating the condition of the patient at the time tB and an index value indicating the condition of the patient at the time tC, when an index value indicating the condition of the patient at the time tA is inputted. The prediction model M is a learnable model (e.g., a machine-learnable model). That is, the prediction model M is a model that updates a parameter for defining the operation of the prediction model M by a learning operation. An example of such a learnable model is a model including a neural network. When the prediction model M is a model including a neural network, the parameter for defining the operation of the prediction model M may include at least one of a structure of the neural network (i.e., a connection aspect of a node), a weight of the neural network, and a bias of the neural network, for example.

The condition of the patient in this example embodiment may mean the condition of the patient that changes over time, for example. An example of the condition of the patient is the condition of a disease of the patient. It is because the condition of the disease of the patient changes due to various factors. For example, the condition of the disease of the patient may change to be better by medical treatment given to the patient. Alternatively, for example, the condition of the disease of the patient may change to be worse even if medical treatment is given to the patient. As an example, if the patient has Alzheimer's disease or the like, the condition of the patient may change to be worse even if medical treatment is given to the patient. Another example of the condition of the patient is at least one of a cognitive function of the patient and a physical function of the patient. It is because at least one of the cognitive function of the patient and the physical function of the patient is improved (i.e., changed) by rehabilitation the patient works on. The example embodiment describes an example in which at least one of the cognitive function of the patient and the physical function of the patient is used as the condition of the patient.

The index value indicating the condition of the patient may mean an index value that changes in accordance with a good or bad condition of the patient. That is, the index value indicating the condition of the patient may mean the index value quantitatively indicating the degree of a good or bad condition of the patient. For example, as the index value, the index value that increases as the condition of the patient is better, may be used. For example, as the index value, the index value that decreases as the condition of the patient is better, may be used. For example, as the index value, the index value that increases as the condition of the patient is worse, may be used. For example, as the index value, the index value that decreases as the condition of the patient is worse, may be used. As described above, when the condition of the patient changes over time, the index value indicating the condition of the patient also changes over time. As described above, the example embodiment describes an example in which at least one of the cognitive function of the patient and the physical function of the patient is used as the condition of the patient. In this case, an index based on Fanctional Independence Measure (FIM) (hereinafter referred to as an “FIM score”) may be used as the index indicating the condition of the patient. Therefore, the example embodiment describes an example in which the FIM score is used as the index value indicating the condition of the patient.

The time tA may mean a time between when the patient is hospitalized and when the patient is discharged from the hospital. The time tB may mean a time that is a predetermined period pB after the patient is discharged from the hospital. The time tC may mean a time that is a predetermined period pC after the patient is discharged from the hospital (wherein the predetermined time period pC is longer than the predetermined time period pB). The example embodiment describes an example in which a time t_D when the patient is hospitalized is used as the time tA, and a time t_F that is half a year after the patient is discharged from the hospital is used as the time tB, and a time t_Y that is a year after the patient is discharged from the hospital is used as the time tC. In this case, the prediction model M is a model that predicts each of an FIM score S_F indicating the condition of the patient at the time t_F and an FIM score S_Y indicating the condition of the patient at the time t_Y, on the basis of an FIM score S_D indicating the condition of the patient at the time t_D. The times tA, tB and tC, however, are not limited to these examples.

Hereinafter, a configuration of the model generation apparatus 1 and the model generation operation performed by the model generation apparatus 1 will be described in order.

### (1-1) Configuration of Model Generation Apparatus 1

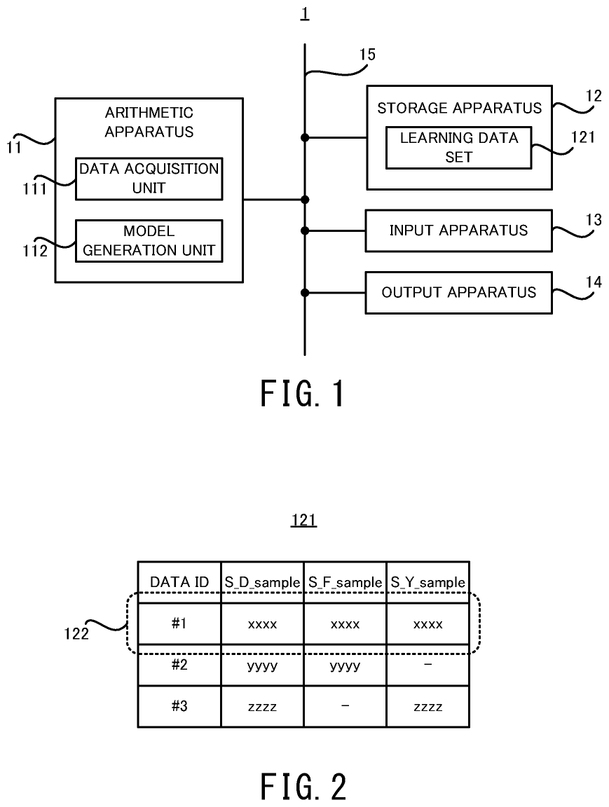

First, the configuration of the model generation apparatus 1 according to the example embodiment will be described with reference to FIG. 1. FIG. 1 is a block diagram illustrating the configuration of the model generation apparatus 1 according to the example embodiment.

As illustrated in FIG. 1, the model generation apparatus 1 includes an arithmetic apparatus 11 and a storage apparatus 12. In addition, the model generation apparatus 1 may include an input apparatus 13 and an output apparatus 14. The model generation apparatus 1, however, may not include at least one of the input apparatus 13 and the output apparatus 14. The arithmetic apparatus 11, the storage apparatus 12, the input apparatus 13, and the output apparatus 14 may be connected through a data bus 15.

The arithmetic apparatus 11 includes, for example, at least one of a CPU (Central Processing Unit), a GPU (Graphical Processing Unit), and a FPGA (Field Programmable Gate Array). The arithmetic apparatus 11 reads a computer program. For example, the arithmetic apparatus 11 may read a computer program stored in the storage apparatus 12. For example, the arithmetic apparatus 11 may read a computer program that is readable by a computer and that is stored by a non-transitory recording medium, by using a not-illustrated recording medium reading apparatus. The arithmetic apparatus 11 may obtain (i.e., download or read) a computer program from a not-illustrated apparatus disposed outside the model generation apparatus 1, through the input apparatus 13 that may function as a communication apparatus. The arithmetic apparatus 11 executes the read computer program. Consequently, a logical functional block for performing an operation to be performed by the model generation apparatus 1 (e.g., the model generation operation described above) is implemented in the arithmetic apparatus 11. That is, the arithmetic apparatus 11 is allowed to function as a controller for implementing a logical functional block for performing the operation to be performed by the model generation apparatus 1.

FIG. 1 illustrates an example of the logical functional block implemented in the arithmetic apparatus 11 for performing the model generation operation. As illustrated in FIG. 1, a data acquisition unit 111 that is a specific example of an “acquisition unit” and a model generation unit 112 that is a specific example of a “learning unit” are implemented in the arithmetic apparatus 11. The operation performed by each of the data acquisition unit 111 and the model generation unit 112 will be described in detail later, but an outline thereof will be described below. The data acquisition unit 111 obtains learning data 122 (see FIG. 2) used to generate the prediction model M, from a learning data set 121 stored by the storage apparatus 12. The model generation unit 112 generates (in other words, learns) the prediction model M by using the learning data 122 obtained by the data acquisition unit 111.

The storage apparatus 12 is configured to store desired data. For example, the storage apparatus 12 may temporarily store a computer program to be executed by the arithmetic apparatus 11. The storage apparatus 12 may temporarily store the data that are temporarily used by the arithmetic apparatus 11 when the arithmetic apparatus 11 executes the computer program. The storage apparatus 12 may store the data that are stored by the model generation apparatus 1 for a long time. The storage apparatus 12 may include at least one of a RAM (Random Access Memory), a ROM (Read Only Memory), a hard disk apparatus, a magneto-optical disk apparatus, a SSD (Solid State Drive) and a disk array apparatus. That is, the storage apparatus 12 may include a non-transitory recording medium.

In the example embodiment, the storage apparatus 12 stores the data that are used by the model generation apparatus 1 to perform the model generation operation. In FIG. 1, the learning data set 121 is illustrated as an example of the data that are used by the model generation apparatus 1 to perform the model generation operation. That is, FIG. 1 illustrates an example in which the storage apparatus 12 stores the learning data set 121.

An example of a data structure of the learning data set 121 is illustrated in FIG. 2. As illustrated in FIG. 2, the learning data set 121 includes a plurality of learning data 122. Each learning datum 122 includes information about the condition of the patient measured in the past. Specifically, each learning datum 122 includes, as the information about the condition of the patient measured in the past, at least one of the FIM score S_D obtained as a result of measuring the condition of the patient at the time t_D, the FIM score S_F obtained as a result of measuring the condition of the patient at the time t_F, and the FIM score S_Y obtained as a result of measuring the condition of the patient at the time t_Y. In this case, the learning data set 121 may include the learning data 122 including all of the FIM score S_D, the FIM score S_F, and the FIM score S_Y (see the learning data 122 with a data ID of #1 in FIG. 2). The learning data set 121 may include the learning data 122 including the FIM score S_D and the FIM score S_F and excluding the FIM score S_Y (see the learning data 122 with a data ID of #2 in FIG. 2). The learning data set 121 may include the learning data 122 including the FIM score S_D and the FIM score S_Y and excluding the FIM score S_F (see the learning data 122 with a data ID of #3 in FIG. 2).

The patient for whom the FIM score is included in the learning data 122, is a patient for whom the FIM score is already measured int the past, and typically, this patient may be different from a patient who is a future target of the estimation of the FIM score by using the prediction model M. For this reason, in the following explanation, if necessary, the patient for whom the FIM score is included in the learning data 122, is referred to as a “sample patient”, and the patient who is a future target of the estimation of the FIM score by using the prediction model M, is referred to as a “target patient”, thereby distinguishing between the two for convenience. The target patient may be the same person as the sample patient. That is, the learning data set 121 may include the learning data 122 including the FIM score measured in the past of the target patient who is a further target of the estimation of the FIM score by using the prediction modeling M.

In the following explanation, if necessary, the FIM score S_D, the FIM score S_F, and the FIM score S_Y included in the learning data 122 are respectively expressed as an FIM score S_D_sample, an FIM score S_F_sample, and an FIM score S_Y_sample, while the FIM score S_F and the FIM score S_Y_predicted by using the prediction model M are respectively expressed as an FIM score S_F_predict and an FIM score S_Y_predict, thereby distinguishing between the two. In this case, the FIM score S_D_sample corresponds to an actual value of the FIM score indicating the condition of the sample patient at the time t_D when the sample patient is hospitalized. In addition, the FIM score S_F_sample corresponds to an actual value of the FIM score indicating the condition of the sample patient at the time t_F that is half a year after the sample patient is discharged from the hospital. In addition, the FIM score S_Y_sample corresponds to an actual value of the FIM score indicating the condition of the sample patient at the time t_Y that is a year after the sample patient is discharged from the hospital. In addition, the FIM score S_F_predict corresponds to a predicted value of the FIM score indicating the condition of the target patient at the time t_F that is half a year after the target patient is discharged from the hospital. The FIM score S_Y_predict corresponds to a predicted value of the FIM score indicating the condition of the target patient at the time t_Y that is a year after the target patient is discharged from the hospital.

The learning data set 121 may include a plurality of learning data 122 each of which includes information about the condition of respective one of a plurality of different sample patients. For example, the learning data set 121 may include (i) first learning data including: the FIM score S_D_sample obtained as a result of measuring the condition of a first sample patient at the time t_D when the first sample patient is hospitalized; and at least one of the FIM score S_F_sample obtained as a result of measuring the condition of the first sample patient at the time t_F that is half a year after the first sample patient is discharged from the hospital, and the FIM score S_Y_sample obtained as a result of measuring the condition of the first sample patient at the time t_Y that is a year after the first sample patient is discharged from the hospital, and (ii) second learning data including: the FIM score S_D_sample obtained as a result of measuring the condition of a second sample patient, who is different from the first sample patient, at the time t_D when the second sample patient is hospitalized; and at least one of the FIM score S_F_sample obtained as a result of measuring the condition of the second patient sample at the time t_F that is half a year after the second sample patient is discharged from the hospital, and the FIM score S_Y_sample obtained as a result of measuring the condition of the second sample patient at the time t_Y that is a year after the second sample patient is discharged from the hospital.

When some sample patient is hospitalized more than once, the learning data set 121 may include the learning data 122 including information about the condition of the sample patient when the sample patient is hospitalized for an N-the time (wherein N is a variable indicating an integer of 1 or more) and the learning data 122 including information about the condition of the same sample patient when the sample patient is hospitalized for an M-th time (wherein M is a variable indicating an integer of 1 or more, but is different from N).

Referring back to FIG. 1, the input apparatus 13 is an apparatus that receives an input of information to the model generation apparatus 1 from the outside of the model generation apparatus 1. For example, the input apparatus 13 may include an operating apparatus (e.g., at least one of a keyboard, a mouse, and a touch panel) that is operable by a user of the model generation apparatus 1. For example, the input apparatus 13 may include a receiving apparatus that is configured to receive information transmitted as data to the model generation apparatus 1, from the outside of the model generation apparatus 1 through a communication network.

The output apparatus 14 is an apparatus that outputs information. For example, the output apparatus 14 may output information about the prediction model M generated by the model generation operation performed by the model generation apparatus 1. As described above, since the prediction model M is used by the condition prediction apparatus 2, the output apparatus 14 may output the information about the prediction model M to the condition prediction apparatus 2. Consequently, the condition prediction apparatus 2 is allowed to predict the condition of the target patient by using the prediction model M. An example of such an output apparatus 14 is a transmission apparatus that is configured to transmit information as data through a communication network or a data bus. The output apparatus 14, however, may include a display (a display apparatus) that is configured to output (i.e., display) information as an image, for example. The output apparatus 14 may include a speaker (an audio output apparatus) that is configured to output information as audio, for example. For example, the output apparatus 14 may include a printer that is configured to output information-printed documents, for example.

### (1-2) Model Generation Operation Performed by Model Generation Apparatus 1

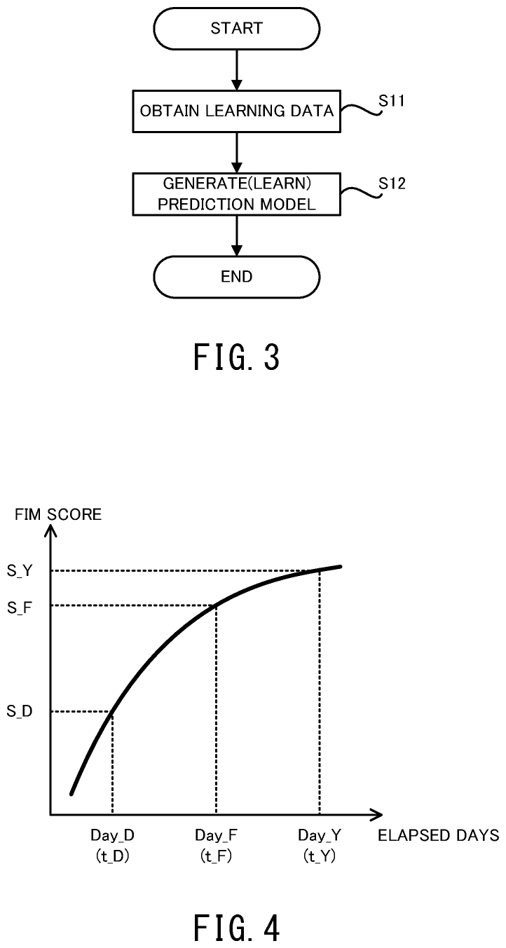

Next, the model generation operation performed by the model generation apparatus 1 will be described with reference to FIG. 3. FIG. 3 is a flowchart illustrating a flow of the model generation operation performed by the model generation apparatus 1.

As illustrated in FIG. 3, first, the data acquisition unit 111 obtains the learning data 122 used to generate the prediction model M, from the learning data set 121 stored in the storage apparatus 12 (step S11). The data acquisition unit 111 obtains all the plurality of learning data 122 included in the learning data set 121. The data acquisition unit 111, however, may obtain a part of the plurality of learning data 122 included in the learning data set 121, but may not obtain another part of the plurality of learning data 122 included in the learning data set 121. For convenience of explanation, the following describes an example in which the data acquisition unit 111 obtains K learning data 122 (specifically, learning data 122#1 to 122#K) (wherein K is a constant indicating an integer of 2 or more).

Then, the model generation unit 112 generates (i.e., learns) the prediction model M by using the plurality of learning data 122 obtained in the step S11 (step S12). Specifically, when the model generation apparatus 1 generates the prediction model M for the first time, the model generation unit 112 inputs the FIM score S_D_sample included in the learning data 122#k obtained in the step S11 (wherein k is a variable indicating an integer satisfying 1≤k≤K), to a default prediction model M (or the prediction model M in an initial state) that is prepared in advance. In the following explanation, the FIM scores S_D_sample, S_F_sample, and S_Y_sample included in the learning data 122#k are respectively expressed as FIM scores S_D_sample#k, S_F_sample#k, and S_Y_sample#k. Alternatively, when the model generation apparatus 1 has already generated the prediction model M, the model generation unit 112 inputs the FIM score S_D_sample#k included in the learning data 122#k obtained in the step S11, to the generated prediction model M. Consequently, the prediction model M outputs the FIM score S_F_predict and the FIM score S_Y_predict. That is, the model generation unit 112 obtains the FIM score S_F_predict and the FIM score S_Y_predict. In the following explanation, the FIM scores S_F_predict and S_Y_predict outputted by the prediction model M to which the FIM score S_D_sample#k is inputted, are respectively expressed as FIM scores S_F_predict#k and S_Y_predict#k.

The model generation unit 112 inputs the FIM score S_D_sample#k to the prediction model M, thereby repeating the operation of obtaining the FIM score S_F_predict#k and the FIM score S_Y_predict#k, for the K learning data 122#1 to 122#K obtained in the step S11. Consequently, the model generation unit 112 obtains FIM scores S_Y_predict#1 to S_F_predict#K and FIM scores S_F_predict#1 to S_Y_predict#K.

Then, the model generation unit 112 updates the default or generated prediction model M on the basis of the FIM scores S_F_predict#1 to S_F_predict#K and the FIM score S_Y_predict#1 to S_Y_predict#K. That is, the model generation unit 112 updates the parameter for defining the operation of the prediction model M, on the basis of the FIM scores S_F_predict#1 to S_F_predict#K and the FIM scores S_Y_predict#1 to S_Y_predict#K. As a result, the updated (i.e., learned) prediction model M is generated. To update the prediction model M, the model generation unit 112 uses a loss function (in other words, an objective function) Loss that is determined on the basis of the FIM score sS_F_predict#1 to S_F_predict#K and the FIM score sS_Y_predict#1 to S_Y_predict#K. That is, the model generation unit 112 generates the prediction model M on the basis of the loss function Loss. Specifically, the model generation unit 112 may update the prediction model M so as to minimize that the loss function Loss, for example.

The loss functional Loss may include a loss term Loss1 that is based on the respective prediction errors of the FIM scores S_F and S_Y. Specifically, the FIM score S_F_predict#k outputted by the prediction model M is a predicted value of the FIM score S_F of the sample patient at the time t_F. On the other hand, the actual value of the FIM score S_F of the sample patient at the time t_F is included as the FIM score S_F_sample#k in the learning data 122#k. Therefore, it is estimated that the prediction accuracy of the prediction model M is improved as a difference between the FIM score S_F_predict#k and the FIM score S_F_sample#k (i.e., the prediction error of the FIM score S_F) is smaller. For the same reason, it is estimated that the prediction accuracy of the prediction model M is improved as a difference between the FIM score S_Y_predict#k and the FIM score S_Y_sample#k (i.e., the prediction error of the FIM score S_Y) is smaller. Therefore, the loss term Loss1 may be a term that is smaller as the difference between the FIM score S_F_predict#k and the FIM score S_F_sample#k is smaller, and/or, as the difference between the FIM score S_Y_predict#k and the FIM score S_Y_sample#k is smaller. An example of such a loss term Loss1 is illustrated in Equations 1 and 2. The Equation 1 indicates an example in which the square error is used as the prediction error. The Equation 2 indicates an example in which the absolute value is used as the prediction error. Considering that the model generation unit 112 generally calculates the loss term Loss1 by performing a matrix operation, the loss term Loss1 illustrated in the Equation 1 is preferably used.

$$\\ \begin{matrix}Loss 1 = {\sum }_{k = 1}^{K} ( \begin{matrix}{( S\_F \_predict \#k - S\_F \_sample \#k )}^{2} + \\ {( S\_Y \_predict \#k - S\_Y \_sample \#k )}^{2}\end{matrix} ) & [ Equation 1 ]\end{matrix}$$

$$\\ \begin{matrix}Loss 1 = {\sum }_{k = 1}^{K} ( \begin{matrix}\vert S\_F \_predict \#k - S\_F \_sample \#k \vert + \\ \vert S\_Y \_predict \#k - S\_Y \_sample \#k \vert\end{matrix} ) & [ Equation 2 ]\end{matrix}$$

Especially in the example embodiment, the loss function Loss includes a loss term Loss2 that is determined on the basis of a change curve C indicating a change tendency of the FIM score over time. In this case, the model generation unit 112 may update the prediction model M so as to minimize the loss functional Loss including the sum of the loss term Loss1 and the loss term Loss2.

The change curve C is information indicating a typical (in other words, general, standard, average or ideal) change tendency of the FIM score over time. Specifically, the condition (in this case, the condition of a disease) of a patient suffering from a certain disease is relatively likely to be better in a tendency that is substantially the same as a tendency in which the condition of the disease of another patient suffering from the same disease is better. That is, the FIM score of a patient suffering from a certain disease is relatively likely to change (typically, increases) in a tendency that is substantially the same as a tendency in which the FIM score of another patient suffering the same disease changes (typically, increases). In other words, the FIM scores of a plurality of patients suffering from the same disease is relatively likely to change in the same way. For example, the above-described Non-Patent literature 1 indicates that the FIM score of a patient suffering from a stroke tends to change along a logarithmic curve.

It can also be said that the change curve C (or any information) indicating a typical change tendency of the FIM score over time, as described above, substantially indicates a predetermined relationship that is expectedly established in the FIM scores S_D_sample#k, S_F_sample#k and S_Y_sample#k included in the learning data 122#k. Then, it is estimated that the prediction accuracy of the prediction model M is improved as the relationship between the FIM score S_D_sample#k inputted to the prediction model M and the FIM scores S_F_predict#k and S_Y_predict#k outputted by the prediction model M, is closer to the predetermined relationship indicated by the change curve C. That is, it is estimated that the prediction accuracy of the prediction model M is improved as a change between the FIM score S_D_sample#k and the FIM scores S_F_predict#k and S_Y_predict#k is closer to the change tendency of the FIM score between the time t_D and the times t_F and t_Y indicated by the change curve C. Therefore, the loss term Loss2 may be a term that is smaller as the relationship among the FIM scores S_D_sample#k, S_F_predict#k, and S_Y_predict#k is closer to the predetermined relationship indicated by the change curve C. That is, the loss term Loss2 may be a term that is smaller as the change between the FIM score S_D_sample#k and the FIM scores S_F_predict#k and S_Y_predict#k is closer to the change tendency of the FIM score between the time t_D and the times t_F and t_Y indicated by the change curve C. In this case, the model generation unit 112 generates the prediction model M such that the change between the FIM score S_D_sample#k and the FIM scores S_F_predict#k and S_Y_predict#k is closer to the change tendency of the FIM score between the time t_D and the times t_F and t_Y indicated by the change curve C.

An example of the loss term Loss2 will be described with reference to FIG. 4. FIG. 4 illustrates an example in which the FIM score is defined by an equation a of S_Day1=S_Day2+β×ln(Day1/Day2). That is, FIG. 4 illustrates an example in which the change curve C indicating the change tendency of the FIM score is expressed by the equation a of S_Day1=S_Day2+β×ln(Day1/Day2). In the equation a, “Day1” and the “Day2” indicate elapsed days from a predetermined reference time (e.g., when a patient develops a disease). “Day1” and the “Day2”, however, are different from each other. In addition, “β” in the equation a means an arbitrary constant. Furthermore, “ln” in the equation a means a logarithm (i.e., a natural logarithm) having the Napier's number e as a base. Therefore, the equation a indicates that a predetermined relationship defined by an equation b of S_Y=S_D+β×ln(Day_Y/Day_D) is established among the FIM scores S_D, S_F, and S_Y. In the equation b, “Day_D” and “Day_Y” respectively indicate elapsed days to the time t_D from the predetermined reference time and elapsed days to the time t_Y from the predetermined reference time. The constant β in the equation b is a constant that can be expressed by equation c of β=S_F−S_D/ln(Day_F/Day_D). In the equation c, “Day_F” indicates elapsed to the time t_F days from the predetermined reference time. The equation b may be replaced by an equation b′ of S_F=S_D+βln (Day_F/Day_D). In this case, the constant β may be expressed by an equation c′ of β=s_Y−S_D/ln (Day_Y/Day_D). Here, when substituting the equation c into the equation b and arranging the equation, the equation b is converted to an equation d of S_Y=S_D×(1−A)+S_F×A. In the equation d, “A” indicates a constant expressed by an equation of A=ln(Day_Y/Day_D)/ln (Day_F/Day_D).

As a consequence, it can be said that the change curve C illustrated in FIG. 4 indicates that a predetermined relationship expressed by the equation d of S_Y=S_D×(1−A)+S_F×A is established among the FIM scores S_D, S_F, and S_Y. That is, the change curve C indicates that the predetermined relationship expressed by the equation d of S_Y_sample=S_D_sample×(1−A)+S_F_sample×A is expectedly established among the FIM scores S_D_sample, S_F_sample and S_Y_sample. Then, considering that the FIM scores S_F_predict and S_Y_predict are respectively the predicted values of the FIM scores S_F_sample and S_Y_sample, it is estimated that the prediction accuracy of the prediction model M is improved as the relationship among the FIM scores S_D_sample, S_F_predict and S_Y_predict is closer to a predetermined relationship indiacted by an equation e of S_Y_predict=S_D_sample8(1−A)+S_F_predict×A. Therefore, the loss term Loss2 may be a term that is smaller as a difference between a left side of the equation e (=S_Y_predict) and a right side of the equation e (=S_D_sample×(1−A)+S_F_predict×A) is smaller. An example of such a loss term Loss2 is illustrated in Equations 3 and 4. The Equation 3 indicates an example in which the squared value of the difference is used. The Equation 4 indicates an example in which the absolute value of the difference is used. Considering that the model generation unit 112 generally calculates the loss term Loss2 by performing a matrix operation, the loss term Loss2 illustrated in Equation 3 is preferably used. The loss term Loss2 may be used as a so-called regularization term.

$$\\ \begin{matrix}Loss 2 = {\sum }_{k = 1}^{K} {( \begin{matrix}S\_Y \_predict \#k - S\_D \_sample \#k \times \\ ( 1 - A ) - S\_F \_predict \#k \times A\end{matrix} )}^{2} & [ Equation 3 ]\end{matrix}$$

$$\\ \begin{matrix}Loss 2 = {\sum }_{k = 1}^{K} \vert \begin{matrix}S\_Y \_predict \#k - S\_D \_sample \#k \times \\ ( 1 - A ) - S\_F \_predict \#k \times A\end{matrix} \vert & [ Equation 4 ]\end{matrix}$$

The loss term Loss2 may be set in advance on the basis of the change curve C. Alternatively, the model generation unit 112 may set the loss term Loss2 on the basis of the change curve C. In either case, it can be said that the model generation unit 112 updates the prediction model M on the basis of the change curve C. That is, it can be said that the model generation unit 112 updates the prediction model M by using prior knowledge about the change curve C.

### (2) Condition Prediction Apparatus 2

Next, the condition prediction apparatus 2 will be described. The condition prediction apparatus 2 performs a condition prediction operation of predicting, on the basis of the condition of the target patient at the time tA, each of the condition of the target patient at the time tB that is after the time tA and the condition of the target patient at the time tC that is after the time tB, by using the prediction model M generated by the model generation apparatus 1. In the example embodiment, as described above, the prediction model M is a model that predicts each of the FIM score S_F indicating the condition of the target patient at the time t_F and the FIM score S_Y indicating the condition of the target patient at the time t_Y that is after the time t_F, on the basis of the FIM score S_D indicating the condition of the target patient at the time t_D. In this case, the condition prediction operation is an operation of predicting each of the FIM score S_F indicating the condition of the target patient at the time t_F and the FIM score S_Y indicating the condition of the target patient at the time t_Y, on the basis of the FIM score S_D indicating the condition of the target patient at the time t_D, by using the prediction model M. Hereinafter, a configuration of the condition prediction apparatus 2 and the condition prediction operation performed by the condition prediction apparatus 2 will be described in order.

### (2-1) Configuration of Condition Prediction Apparatus 2

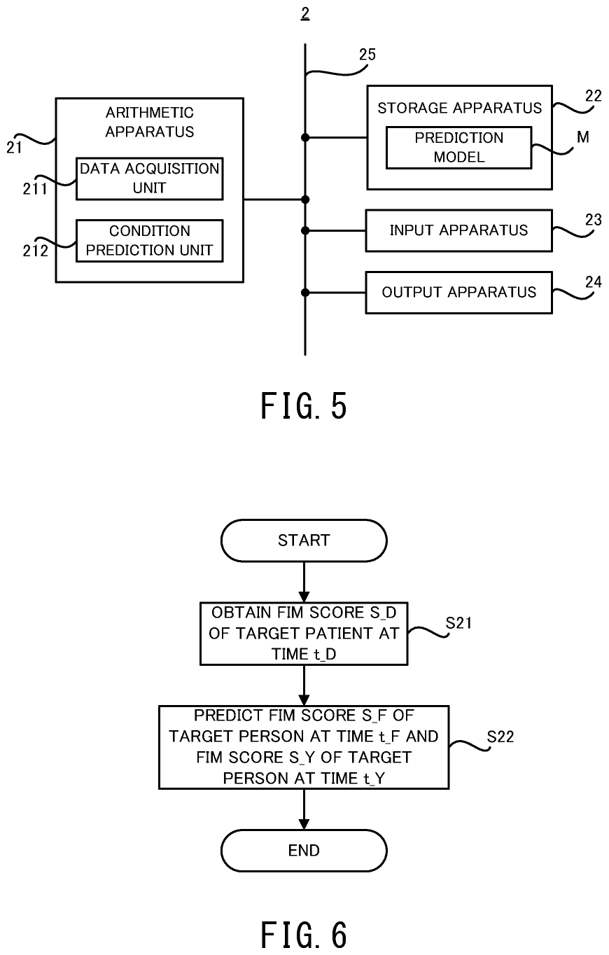

First, with reference to FIG. 5, the configuration of the condition prediction apparatus 2 according to the example embodiment will be described. FIG. 5 is a block diagram illustrating the configuration of the condition prediction apparatus 2 according to the example embodiment.

As illustrated in FIG. 5, the condition prediction apparatus 2 includes an arithmetic apparatus 21 and a storage apparatus 22. Furthermore, the condition prediction apparatus 2 may include an input apparatus 23 and an output apparatus 24. The condition prediction apparatus 2, however, may not include at least one of the input apparatus 23 and the output apparatus 24. The arithmetic apparatus 21, the storage apparatus 22, the input apparatus 23, and the output apparatus 24 may be connected through a data bus 25.

The arithmetic apparatus 21 includes, for example, at least one of a CPU, a GPU and a FPGA. The arithmetic apparatus 21 reads a computer program. For example, the arithmetic apparatus 21 may read a computer program stored in the storage apparatus 22. For example, the arithmetic apparatus 21 may read a computer program that is readable by a computer and that is stored by a non-transitory recording medium, by using a not-illustrated recording medium reading apparatus. The arithmetic apparatus 21 may obtain (i.e., download or read) a computer program from a not-illustrated apparatus disposed outside the condition prediction apparatus 2, through the input apparatus 23 that may function as a communication apparatus. The arithmetic apparatus 21 executes the read computer program. Consequently, a logical functional block for performing an operation to be performed by the condition prediction apparatus 2 (e.g., the condition prediction operation described above) is implemented in the arithmetic apparatus 21. That is, the arithmetic apparatus 21 is allowed to function as a controller for implementing a logical functional block for performing the operation to be performed by the condition prediction apparatus 2.

FIG. 5 illustrates an example of the logical functional block implemented in the arithmetic apparatus 21 for performing the condition prediction operation. As illustrated in FIG. 5, a data acquisition unit 211 that is a specific example of the “acquisition unit” and a condition prediction unit 212 that is a specific example of a “prediction unit” are implemented in the arithmetic apparatus 21. The operation performed by each of the data acquisition unit 211 and the condition prediction unit 212 will be described in detail later, but an outline thereof will be described below. The data acquisition unit 211 obtains the FIM score S_D indicating the condition of the target patient at the time t_D. The condition prediction unit 212 predicts each of the FIM score S_F indicating the condition of the target patient at the time t_F and the FIM score S_Y indicating the condition of the target patient at the time t_Y, by using the prediction model M and the FIM score S_D obtained by the data acquisition unit 211.

The storage apparatus 22 is configured to store desired data. For example, the storage apparatus 22 may temporarily store a computer program to be executed by the arithmetic apparatus 21. The storage apparatus 22 may temporarily store the data that are temporarily used by the arithmetic apparatus 21 when the arithmetic apparatus 21 executes the computer program. The storage apparatus 22 may store the data that are stored by the condition prediction apparatus 2 for a long time. For example, in the example illustrated in FIG. 5, the storage apparatus 22 stores the prediction model M used by the condition prediction apparatus 2. The storage apparatus 22 may include at least one of a RAM, a ROM, a hard disk apparatus, a magneto-optical disk apparatus, a SSD and a disk array apparatus. That is, the storage apparatus 22 may include a non-transitory recording medium.

The input apparatus 23 is an apparatus that receives an input of information to the condition prediction apparatus 2 from the outside of the condition prediction apparatus 2. For example, the input apparatus 23 may include an operating apparatus (e.g., at least one of a keyboard, a mouse, and a touch panel) that is operable by a user of the condition prediction apparatus 2. For example, the input apparatus 23 may include a receiving apparatus that is configured to receive information transmitted as data to the condition prediction apparatus 2, from the outside of the condition prediction apparatus 2 through a communication network.

The output apparatus 24 is an apparatus that outputs information. For example, the output apparatus 24 may output information about a prediction result of the condition prediction apparatus 2 (e.g., each of the FIM scores S_F and S_Y). An example of such an output apparatus 24 is a transmission apparatus that is configured to transmit information as data through a communication network or a data bus. The output apparatus 24, however, may include a display (a display apparatus) that is configured to output (i.e., display) information as an image, for example. The output apparatus 24 may include a speaker (an audio output apparatus) that is configured to output information as audio, for example. For example, the output apparatus 24 may include a printer that is configured to output information-printed documents, for example.

### (2-2) Condition Prediction Operation Performed by Condition Prediction Apparatus 2

Next, the condition prediction operation performed by the condition prediction apparatus 2 will be described with reference to FIG. 6. FIG. 6 is a flowchart illustrating a flow of the condition prediction operation performed by the condition prediction apparatus 2.

As illustrated in FIG. 6, first, the data acquisition unit 211 obtains the FIM score S_D indicating the condition of the target patient at the time t_D (step S21). For example, when the FIM score S_D is stored in the storage apparatus 22, the data acquisition unit 211 may obtain the FIM score S_D from the storage apparatus 22. For example, when the tFIM score S_D is inpuedt to the condition prediction apparatus 2 through the input apparatus 23, the data acquisition unit 211 may obtain the FIM score S_D by using the input apparatus 23.

Then, the condition prediction unit 212 predicts each of the FIM score S_F indicating the condition of the target patient at the time t_F and the FIM score S_Y indicating the condition of the target patient at the time t_Y, by using the prediction model M and the FIM score S_D obtained in the step S21 (step S22). Specifically, the condition prediction unit 212 inputs the FIM score S_D obtained in the step S21, to the prediction model M. Consequently, the prediction model M outputs the FIM score S_F_predict and the FIM score S_Y_predict. That is, the condition prediction unit 212 obtains the FIM score S_F_predict from the prediction model M, as the predicted value of the FIM score S_F indicating the condition of the target patient at the time t_F.

Furthermore, the condition prediction unit 212 obtains the FIM score S_Y_predict from the prediction model M, as the predicted value of the FIM score S_Y indicating the condition of the target patient at the time t_Y.

### (3) Technical Effect

As described above, the model generation apparatus 1 according to the example embodiment generates the prediction model M on the basis of the change curve C (i.e., information indicating the change tendency of the condition of the patient). That is, the model generation apparatus 1 generates the prediction model M by using the loss function Loss including the loss term Loss2 that is determined on the basis of the change curve C. Therefore, the model generation apparatus 1 is capable of generating the prediction model M that allows the output of the prediction result that is relatively likely to match the change tendency of the condition of the patient indicated by the change curve C, in comparison with a model generation apparatus according to a comparative example. The model generation apparatus according to the comparative example is an apparatus that generates the prediction model M by using a loss function excluding the loss term Loss2 (e.g., a loss function including the loss term Loss1, but excluding the loss term Loss2). Therefore, the model generation apparatus 1 is allowed to properly improve the prediction accuracy of the prediction model M.

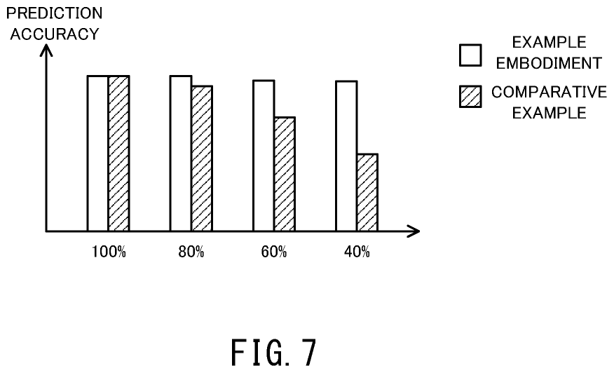

In addition, in the example embodiment, the model generation apparatus 1 generates the prediction model M that predicts the FIM scores S_F and S_Y indicating the condition of the target patient at the times t_F and t_Y after the target patient is discharged from the hospital. Here, as described above, when the loss function Loss includes the loss term Loss1 regarding the prediction error, the prediction accuracy of the FIM scores S_F and S_Y by the prediction model M is improved with increasing the number of samples of the FIM scores S_F_sample and S_Y_sample indicating the condition of the patient at the times t_F and t_Y after the sample patient is discharged from the hospital. It is, however, not always easy to measure the condition of the patient once the patient is discharged from the hospital, compared to the case of measuring the condition of the patient while the patient is in the hospital. This is because it is not easy to encourage the patient who is discharged from the hospital, to visit the hospital to measure the condition. Therefore, it is not always easy to collect a large number of the FIM scores S_F_sample and S_Y_sample. Meanwhile, the model generation apparatus 1 according to the example embodiment generates the prediction model M, by using the loss function Loss including the loss term Loss2 based on the change curve C, in addition to or in place of the loss term Loss1 regarding the prediction error. Therefore, the model generation apparatus 1 according to the example embodiment is allowed to properly improve the prediction accuracy of the prediction model M, even when the sample number of the FIM scores S_F_sample and S_Y_sample is relatively low, in comparison with the model generation apparatus according to the comparative example. For example, FIG. 7 illustrates the prediction accuracy of the prediction model M generated by the model generation apparatus 1 according to the example embodiment and the prediction accuracy of the prediction model M generated by the model generation apparatus according to the comparative example. A horizontal axis in FIG. 7 indicates the ratio of a total number of the sample patients for whom the FIM scores S_F_sample and S_Y_sample at the times t_F and t_Y are measurable, with respect to a total number of the sample patients for whom the FIM score S_D_sample at the time t_D is already measured. Specifically, “X%” indicated by the horizontal axis in FIG. 7 indicates that only the FIM score S_F_sample and S_Y_sample of X×Y sample patients are measurable (conversely, the FIM scores S_F_sample and S_Y_sample of (1−X)×Y sample patients are not measurable) in the situation where the FIM score S_D_sample of Y sample patients is already measured. As illustrated in FIG. 7, the predictive accuracy of the prediction model M generated by the model generation apparatus according to the comparative example is worsen as the sample number of the FIM scores S_F_sample and S_Y_sample is reduced (i.e., with reducing the ratio of the total number of the sample patients for whom the FIM scores S_F_sample and S_Y_sample are measurable). On the other hand, the prediction accuracy of the prediction model M generated by the model generation apparatus 1 according to the example embodiment is not worsen as much as the prediction accuracy of the prediction model M generated by the model generation apparatus according to the comparative example, even with reducing the sample number of the FIM scores S_F_sample and S_Y_sample.

In addition, the condition prediction apparatus 2 according to the example embodiment predicts the condition of the patient by using the prediction model M generated by the model generation apparatus 1 according to the example embodiment. Therefore, the condition prediction apparatus 2 is allowed to properly predict the condition of the patient.

### (4) Modified Example

In the above-described description, the prediction model M is a model that predicts, on the basis of the condition of the patient at the time tA (e.g., the time t_D when the patient is hospitalized), each of the condition of the patient at the time tB that is after the time tA (e.g., the time t_F that is half a year after the patient is discharged from the hospital) and the condition of the patient at the time tC that is after the time tB (e.g., the time t_Y that is a year after the patient is discharged from the hospital). The prediction model M, however, may be a model that predicts the condition of the patient at one of the times tB and tC, but that does not predict the condition of the patient at the other of the times tB and tC, on the basis of the condition of the patient at the time tA. Even in this case, the model generation apparatus 1 may generate the prediction model M by performing the model generation operation described above.

As a modified example, a description will be given to a model generation operation for generating the prediction model M that predicts the FIM score S_Y indicating the condition of the patient at the time t_Y on the basis of the FIM score S_D indicating the condition of the patient at the time t_D. Even in the modified example, the data acquisition unit 111 obtains the learning data 122 (step S11 in FIG. 3). In the modified example, however, the learning data 122 may not include the FIM scoring S_F_sample indicating the condition of the sampler at the time t_F. Then, the model generation unit 112 obtains the FIM score S_Y_predict#k by inputting the FIM score S_D_sample#k to the prediction model M. Then, the model generation unit 112 updates the prediction model M so as to minimize the loss function Loss, on the basis of the FIM scores score S_Y_predict#1 to S_Y_predict#K. In the modified example, however, the loss term Loss1 is a term that is smaller as the difference between the FIM score S_Y_predict#k and the FIM score S_Y_sample#k is smaller. An example of such a loss term Loss1 is illustrated in Equations 5 and 6. In addition, in the modified example, the loss term Loss2 may be a term that is smaller as the relationship between the FIM scores S_D_sample#k and S_Y_predict#k is closer to the predetermined relationship indicated by the change curve C. An example of such a loss term Loss2 is illustrated in Equations 6 and 7. In the modified example, however, the constant β is preferably known to the model generation apparatus 1 (i.e., a preset value).

$$\\ \begin{matrix}Loss 1 = {\sum }_{k = 1}^{K} {( S\_Y \_predict \#k - S\_Y \_sample \#k )}^{2} & [ Equation 5 ]\end{matrix}$$

$$\\ \begin{matrix}Loss 1 = {\sum }_{k = 1}^{K} \vert S\_Y \_predict \#k - S\_Y \_sample \#k \vert & [ Equation 6 ]\end{matrix}$$

$$\\ \begin{matrix}Loss 2 = {\sum }_{k = 1}^{K} {( S\_Y \_predict \#k - S\_D \_sample \#k - β \times ln ( \frac{Day\_Y}{Day\_D} ) )}^{2} & [ Equation 7 ]\end{matrix}$$

$$\\ \begin{matrix}Loss 2 = {\sum }_{k = 1}^{K} \vert S\_Y \_predict \#k - S\_D \_sample \#k - β \times ln ( \frac{Day\_Y}{Day\_D} ) \vert & [ Equation 8 ]\end{matrix}$$

By replacing the reference numeral “Y” in the description of the model generation operation in the modified example by the reference numeral “F”, the description of the model generation operation described above is changed to a description of a model generation operation for generating the prediction model M that predicts the FIM score S_F indicating the condition of the patient at the time t_F, on the basis of the FIM score S_D indicating the condition of the patient at the time t_D.

### (5) Supplementary Notes

With respect to the example embodiments described above, the following Supplementary Notes are further disclosed.

### [Supplementary Note 1]

A model generation apparatus including:
an acquisition unit that is configured to obtain learning data including a first index value indicating a condition of a sample patient at a first time, and a second index value indicating a condition of the sample patient at a second time that is after the first time; and
a learning unit that is configured to learn a prediction model for predicting a condition of a target patient at the second time on the basis of a condition of the target patient at the first time, wherein
the learning unit is configured to learn the prediction model on the basis of a third index value indicating a condition of the sample patient at the second time that is predicted by the prediction model on the basis of the first index value, and a change information indicating a change tendency of the condition over time.

### [Supplementary Note 2]

The model generation apparatus according to Supplementary Note 1, wherein the learning unit is configured to learn the prediction model such that a change between the first and third index values is closer to a first change tendency that is a change tendency of the condition from the first time to the second time indicated by the change information.

### [Supplementary Note 3]

The model generation apparatus according to Supplementary Note 2, wherein the learning unit is configured to learn the prediction model on the basis of a loss function including a term that is smaller as the change between the first and third index values is closer to the first change tendency.

### [Supplementary Note 4]

The model generation apparatus according to any one of Supplementary Notes 1 to 3, wherein
the first time is a time between a time at which a patient including the sample patient and the target patient is hospitalized and a time at which the patient is discharged from the hospital, and
the second time is a time that is a first predetermined period after the patient is discharged from the hospital.

### [Supplementary Note 5]

The model generation apparatus according to any one of Supplementary Notes 1 to 4, wherein
the prediction model further predicts a condition of the target patient at a third time that is after the first time and that is different from the second time,
the learning data includes a fourth index value indicating a condition of the sample patient at the third time, and
the learning unit is configured to learn the prediction model on the basis of the third index value, a fifth index value indicating a condition of the sampler at the third time that is predicted by the prediction model on the basis of the first index value, and the change information.

### [Supplementary Note 6]

The model generation apparatus according to Supplementary Note 5, wherein the learning unit is configured to learn the prediction model such that a change among the first index value, the third index value, and the fifth index value is closer to a second change tendency of the condition from the first time to the second time and the third time indicated by the change information.

### [Supplementary Note 7]

The model generation apparatus according to Supplementary Note 6, wherein the learning unit is configured to learn the prediction model on the basis of a loss function including a term that is smaller as the change among the first index value, the third index value, and the fifth index value is closer to the second change tendency.

### [Supplementary Note 8]

The model generation apparatus according to any one of Supplementary Notes 5 to 7, wherein
the first time is a time between a time at which a patient including the sample patient and the target patient is hospitalized and a time at which the patient is discharged from the hospital,
the second time is a time that is a first predetermined period after the patient is discharged from the hospital, and
the third time is a time that is a second predetermined time after the patient is discharged from the hospital, wherein the second predetermined time is different from the first predetermined time.

### [Supplementary Note 9]

The model generation apparatus according to Supplementary Note 4 or 8, wherein the first time is a time when the patient is hospitalized.

### [Supplementary Note 10]

A model generation method including:
obtaining learning data including a first index value indicating a condition of a sample patient at a first time, and a second index value indicating a condition of the sample patient at a second time that is after the first time; and
learning a prediction model for predicting a condition of a target patient at the second time on the basis of a condition of the target patient at the first time, wherein
in the learning of the learning, the prediction model is learned on the basis of a third index value indicating a condition of the sample patient at the second time that is predicted by the prediction model on the basis of the first index value, and a change information indicating a change tendency of the condition over time.

### [Supplementary Note 11]

A recording medium on which a computer program that allows a computer to execute a model generation method is recorded, the model generation method including:
obtaining learning data including a first index value indicating a condition of a sample patient at a first time, and a second index value indicating a condition of the sample patient at a second time that is after the first time; and
learning a prediction model for predicting a condition of a target patient at the second time on the basis of a condition of the target patient at the first time, wherein
in the learning of the learning, the prediction model is learned on the basis of a third index value indicating a condition of the sample patient at the second time that is predicted by the prediction model on the basis of the first index value, and a change information indicating a change tendency of the condition over time.

### [Supplementary Note 12]

A computer program that allows a computer to execute a model generation method, the model generation method including:
obtaining learning data including a first index value indicating a condition of a sample patient at a first time, and a second index value indicating a condition of the sample patient at a second time that is after the first time; and
learning a prediction model for predicting a condition of a target patient at the second time on the basis of a condition of the target patient at the first time, wherein
in the learning of the learning, the prediction model is learned on the basis of a third index value indicating a condition of the sample patient at the second time that is predicted by the prediction model on the basis of the first index value, and a change information indicating a change tendency of the condition over time.

### [Supplementary Note 13]

A condition prediction apparatus including:
a condition acquisition unit that is configured to obtain a condition information about a condition of a target patient at a first time; and
a prediction unit that is configured to predict a condition of the target patient at a second time that is after the first time, by using the condition information and a prediction model for predicting a condition of the target patient at the second time on the basis of the condition of the target patient at the first time, wherein
the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

### [Supplementary Note 14]

A condition prediction method including:
obtaining a condition information about a condition of a target patient at a first time; and
predicting a condition of the target patient at a second time that is after the first time, by using the condition information and a prediction model for predicting a condition of the target patient at the second time on the basis of the condition of the target patient at the first time, wherein
the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

### [Supplementary Note 15]

A recording medium on which a computer program that allows a computer to execute a condition prediction method is recorded, the condition prediction method including:
obtaining a condition information about a condition of a target patient at a first time; and
predicting a condition of the target patient at a second time that is after the first time, by using the condition information and a prediction model for predicting a condition of the target patient at the second time on the basis of the condition of the target patient at the first time, wherein
the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

### [Supplementary Note 16]

A computer program that allows a computer to execute a condition prediction method, the condition prediction method including:
obtaining a condition information about a condition of a target patient at a first time; and
predicting a condition of the target patient at a second time that is after the first time, by using the condition information and a prediction model for predicting a condition of the target patient at the second time on the basis of the condition of the target patient at the first time, wherein
the prediction model is a model generated by obtaining learning data including a first index value indicating a condition of a sample patient at the first time, and a second index value indicating a condition of the sample patient at the second time, and performing learning on the basis of a third index value indicating a condition of the sample patient at the second time that is predicted by the prediction model on the basis of the first index value, and a change information indicating a change tendency of the condition over time.

This disclosure is not limited to the examples described above and is allowed to be changed, if desired, without departing from the essence or spirit of this disclosure which can be read from the claims and the entire specification. A parameter determination apparatus, a parameter determination method, a signal transmission method, and a computer program with such changes are also intended to be within the technical scope of this disclosure.

### DESCRIPTION OF REFERENCE CODES

1 Model generation apparatus
11 Arithmetic apparatus
111 Data acquisition unit
112 Model generation unit
12 Storage apparatus
121 Learning data set
122 Learning data
2 Condition prediction apparatus
21 Arithmetic apparatus
211 Data acquisition unit
212 Condition prediction unit
M Prediction model

What is claimed is:

1. A model generation apparatus comprising:
at least one memory configured to store instructions; and
at least one processor configured to execute the instructions to:
to obtain learning data including a first index value indicating a condition of a sample patient at a first time, and a second index value indicating a condition of the sample patient at a second time that is after the first time; and
learn a prediction model for predicting a condition of a target patient at the second time on the basis of a condition of the target patient at the first time, wherein
the at least one processor configured to execute the instructions to learn the prediction model so as to minimize a loss function including a term that is smaller as the change between the first index value and a third index is closer to a first change tendency, wherein the third index value indicating-indicates a condition of the sample patient at the second time that is predicted by the prediction model on the basis of the first index value, and the first change tendency indicates a change tendency of the condition from the first time to the second time.

2-3. (canceled)

4. The model generation apparatus according to claim 1, wherein
the first time is a time between a time at which a patient including the sample patient and the target patient is hospitalized and a time at which the patient is discharged from the hospital, and
the second time is a time that is a first predetermined period after the patient is discharged from the hospital.

5. The model generation apparatus according to claim 1, wherein
the prediction model further predicts a condition of the target patient at a third time that is after the first time and that is different from the second time,
the learning data includes a fourth index value indicating a condition of the sample patient at the third time, and
the at least one processor configured to execute the instructions to learn the prediction model on the basis of the third index value, a fifth index value indicating a condition of the sampler at the third time that is predicted by the prediction model on the basis of the first index value, and change information indicating a change tendency of the condition over time.

6. The model generation apparatus according to claim 5, wherein
the at least one processor configured to execute the instructions to learn the prediction model such that a change among the first index value, the third index value, and the fifth index value is closer to a second change tendency of the condition from the first time to the second time and the third time indicated by the change information.

7. The model generation apparatus according to claim 6, wherein
the at least one processor configured to execute the instructions to learn the prediction model on the basis of a loss function including a term that is smaller as the change among the first index value, the third index value, and the fifth index value is closer to the second change tendency.

8. The model generation apparatus according to claim 5, wherein
the first time is a time between a time at which a patient including the sample patient and the target patient is hospitalized and a time at which the patient is discharged from the hospital,
the second time is a time that is a first predetermined period after the patient is discharged from the hospital, and
the third time is a time that is a second predetermined time after the patient is discharged from the hospital, wherein the second predetermined time is different from the first predetermined time.

9. The model generation apparatus according to claim 4, wherein
the first time is a time when the patient is hospitalized.

10. A model generation method comprising:
obtaining learning data including a first index value indicating a condition of a sample patient at a first time, and a second index value indicating a condition of the sample patient at a second time that is after the first time; and
learning a prediction model for predicting a condition of a target patient at the second time on the basis of a condition of the target patient at the first time, wherein
in the learning of the learning, the prediction model is learned so as to minimize a loss function including a term that is smaller as the change between the first index value and a third index is closer to a first change tendency, wherein the third index value indicates a condition of the sample patient at the second time that is predicted by the prediction model on the basis of the first index value, and the first change tendency indicates a change tendency of the condition from the first time to the second time.

11. (canceled)

12. A condition prediction apparatus comprising:
at least one memory configured to store instructions; and
at least one processor configured to execute the instructions to:
obtain a condition information about a condition of a target patient at a first time; and
predict a condition of the target patient at a second time that is after the first time, by using the condition information and a prediction model for predicting a condition of the target patient at the second time on the basis of the condition of the target patient at the first time, wherein
the prediction model is a model generated by obtaining learning data including a first index value indicating a condition of a sample patient at the first time, and a second index value indicating a condition of the sample patient at the second time, and performing learning so as to minimize a loss function including a term that is smaller as the change between the first index value and a third index is closer to a first change tendency, wherein the third index value indicates a condition of the sample patient at the second time that is predicted by the prediction model on the basis of the first index value, and the first change tendency indicates a change tendency of the condition from the first time to the second time.

13-14. (canceled)
